(19) United States
(12) Patent Application Publication
(10) Pub. No.: US 2023/0241410 A1
(43) Pub. Date: Aug. 3, 2023

(54) ADJUSTABLE THERAPY SPECTACLES FOR DECREASING MYOPIA PROGRESSION

(71) Applicant: ACUCELA INC., Seattle (US)

(72) Inventors: Ryo KUBOTA, Seattle, WA (US); Benjamin CROOK, Bremgarten bei Bern (CH); Stefan BAUER, Bern (CH); Philip M. BUSCEMI, Mount Pleasant, SC (US); Amitava GUPTA, Seattle, WA (US); Jonas GERMANN, Gumligen (CH)

(73) Assignee: ACUCELA INC., Seattle (US)

(21) Appl. No.: 18/163,376

(22) Filed: Feb. 2, 2023

Related U.S. Application Data
(60) Provisional application No. 63/365,400, filed on May 26, 2022.
(60) Provisional application No. 63/268,166, filed on Feb. 17, 2022.
(60) Provisional application No. 63/267,515, filed on Feb. 3, 2022.

(51) Int. Cl. A61N 5/06

(52) U.S. Cl. CPC A61N 5/0622; A61N 2005/0648; A61N 2005/0651

(57) ABSTRACT

A wearer adjustable apparatus is configured for a wearer to view an object and adjust stimuli projected into the eye by adjusting the location of the projection optics, for example with movement of the projection optics relative to one or more components of a support such as an eyeglass frame. In some embodiments, the projection system comprises an adjustable retainer for the wearer to adjust first active projection elements coupled to a first support in front of a first eye in relation to second projection optics coupled to a second support in front of a second eye of the wearer. In some embodiments, the apparatus comprises oculars comprising the projection optics and corrective lenses configured for the wearer to adjust positions of the lenses and projection optics in front of the wearer's eyes in order to project therapeutic spots onto a desired location relative to the wearer's retinas.

### RELATED APPLICATIONS

This application claims the benefit under 35 U.S.C. § 119(e) of U.S. Provisional Patent Application No. 63/365,400, filed May 26, 2022, of U.S. Provisional Application No. 63/268,166, filed Feb. 17, 2022, and of U.S. Provisional Patent Application No. 63/267,515, filed Feb. 3, 2022, the entire disclosures of which are incorporated herein by reference.

The subject matter of this patent application is related U.S. patent application Ser. No. 16/947,537, filed Aug. 5, 2020, now U.S. Pat. No. 11,358,001, issued Jun. 14, 2022, U.S. patent application Ser. No. 17/304,691, filed Jun. 24, 2021, now U.S. Pat. No. 11,275,259, issued Mar. 15, 2022, and U.S. patent application Ser. No. 17/303,889, filed Jun. 9, 2021, now U.S. Pat. No. 11,281,022, issued Mar. 22, 2022, the entire disclosures of which are incorporated herein by reference.

### BACKGROUND

Myopia, also known as near-sightedness, is a visual disorder that is frequently progressive and worsens through adolescence and early adulthood. It is characterized by the ability to see objects clearly at nearer distances, but at farther distances objects become blurry. This is primarily the result of the eye being a non-spherical shape and instead being elongated, so that images of objects are not focused at the retina. In some myopic persons, the axial dimension or axis of the eye increases over time, with the result of worsening near-sightedness. The elongated dimension of the eye is commonly referred to as its axial length and is measured along the primary visual axis.

It has been demonstrated that the progression of myopia in some patients can be slowed or stopped by providing stimuli consisting of an image that is defocused in front of the retina, a technique referred to as myopic defocus. Some prior approaches for providing such stimuli rely on passive manipulation of light from the surrounding environment. However, this approach can lead to less than ideal visual results and degraded image quality in at least some instances. One approach that has been suggested involves the projection of stimulus or stimuli into the mid peripheral area of the retina, where the stimulus image is then myopically defocused. However, at least some of the prior approaches can be somewhat more complex and less than ideally suited for use with vision devices such as spectacles. Work in relation to the present disclosure suggests that alignment of such devices can be less than ideal in at least some instances. For example, the projection optics and light stimuli of such devices may be less than ideally aligned with the eyes of the subject, which can provide less than ideal results. Work in relation to the present disclosure suggests that improper alignment of one or more stimuli with respect to the eye can result in the iris occluding at least a portion of the beam, such that the stimulus has a decreased intensity and may not be suitably visible in at least some instances.

In light of the above improved methods and apparatus are needed that ameliorate at least some of the aforementioned limitations of the prior approaches. Ideally, such methods and apparatus would provide improved alignment of light sources and associated stimuli with the eyes of the subject.

### SUMMARY

Embodiments of the present disclosure are directed to apparatuses and methods for a wearer adjustable active projection apparatus for use in the treatment of refractive error, which may be incorporated into spectacles, contact lenses, Augmented Reality (AR) devices or Virtual Reality (VR) devices, or provided as an add-on layer or film to existing spectacles, lenses, AR devices, or VR devices. In some embodiments, the wearer adjustable apparatus is configured for a wearer to view an object and adjust the location of stimuli projected into the eye by adjusting the location of the projection optics that direct the stimulus into the eye, which can provide the stimuli to the retina with decreased interference from the pupil and improve therapeutic results. The stimulus can be directed to any suitable location of the retina and can be focused in front of the retina or virtually behind the retina. In some embodiments, an adjustable retainer is configured to adjust placement of the projection optics on each side of a nose of a wearer in nasal-temporal directions to adjust nasal-temporal locations of the plurality of stimuli on the wearer's retinas. In some embodiments, nose pads coupled to the plurality of projection optics are adjustable to allow the wearer to adjust inferior-superior placement of the projection optics and corresponding inferior-superior locations of the plurality of stimuli on the wearer's retinas. In some embodiments, the apparatus comprises temple portions configured for the wearer to adjust a vertex distance between a vertex of the cornea of the eye and corresponding location of the projection optics. These wearer adjustable components can allow the user to customize the placement of the apparatus with respect to the eyes of the wearer to provide improved placement of the stimuli and therapeutic benefits.

While the wearer can adjust the optics in many ways, in some embodiments, the wearer adjusts the optics in response to an intensity of a perceived stimulus to decrease dimming or truncation of the stimulus that can result from partial or complete occlusion the projected light beams by the iris. In some embodiments, the locations of the stimuli projected onto the retinas of each of the eyes are adjusted by moving the projection optics relative to the eyes of the wearer, such as with movement of the projection optics relative to each other or one or more components of a support such as an eyeglass frame to appropriately align the projection optics with the eyes of the wearer.

In some embodiments, the stimuli projected onto the retina of each eye are adjusted by moving the projection optics automatically, based on information on the location of the pupillary center of each eye obtained by using an onboard eye tracker. In some embodiments, adjustment of the projection optics allows the optical bundle of light rays from one or more stimuli to pass through the pupil of the eye to a position focused anterior to the retina, or virtually posterior to the retina or on the retina with decreased interference from the iris. In some embodiments one or more eye trackers is mounted on one or more oculars of each frame. In some embodiments, one or more eye trackers is mounted on one or both hinges of the eye glass frame. In one embodiment, the location of the pupillary center is determined by monitoring one or more Purkinje images generated by eye in response to a near infrared light signal generated by the eye tracker.

In some embodiments, the projection optic may be directed to form an image such as a defocused image on the foveola, or the macula, or the parafovea or the perifovea. In some embodiments, the image is focused anterior to the retina, or virtually posterior to the retina, resulting in a defocused image on the retina.

In some embodiments, the projection optic may comprise one or more image forming means that are imaged by multiple focusing means, each such image being projected at a specified location of the retina. In some embodiments, the projection of images may utilize free space, or a light pipe or an optical fiber. When multiple projection means are used to project multiple images on the retina, such projection means are required to be phase matched, in order to match the optical path length of all such projection means.

In some embodiments, the projection apparatus comprises an adjustable retainer for the wearer to adjust the active projection elements in relation to one or more components of a support such as an eyeglass frame. In some embodiments, the apparatus comprises oculars comprising the projection optics and corrective lenses configured for the wearer to adjust positions of the lenses and projection optics in front of the wearer's eyes to project therapeutic spots onto the retinas of the wearer's eyes at appropriate locations. In some embodiments, the adjustable retainer allows the wearer to adjust the placement of the projection optics while the wearer views an object and the one or more stimuli in response to an intensity of the one or more stimuli in order to decrease interference from the pupil. In some embodiments, the adjustable retainer allows the wearer to adjust the placement of the plurality of stimuli on the retina while the wearer views an object and adjusts the projection optics. In some embodiments, the adjustable retainer is configured to retain the placement of the lenses and projection optics at respective locations in front of the wearer's eyes. While the wearer adjustable retainer can be configured in many ways, in some embodiments the retainer comprises one or more of a slide, a rail, a slot, a pivot, a hinge, a living hinge, an extension and a guide to receive the extension, a telescopic mechanism, or a rack, pinion, a screw, a thumb screw, a friction pad, a brake, a hole, an extension, a guide to receive an extension, a cam, or a detent.

In some embodiments, the wearer adjustable apparatus comprises an adjustable retainer configured for a wearer to view an object and adjust the location of stimuli projected into the eye by adjusting the location of the projection optics of the stimulus, such as with of a first lens support and a second lens support, in which the supports comprise one or more components of an eyeglass frame. In some embodiments, the adjustable retainer comprises an adjustable bridge that couples a first lens and a first plurality of projection modules on a first support to a second support that supports a second lens and a second plurality of projection modules, in which the adjustable bridge is configured to allow the wearer to move the first plurality of projection modules of the first support relative to the second plurality of projection modules of the second support to position the stimulus from the first support and the stimulus from the second support at respective locations of respective retinas of the eyes of the wearer to decrease the progression. The adjustable bridge may be configured to independently move the first and second supports relative to the centerline of the wearer adjustable apparatus.

In some embodiments, a wearer adjustable apparatus for an active projection apparatus for treatment of refractive error comprises a plurality of projection modules which are coupled together with a common movable support and configured to move together with the common support in front of an eye of the wearer while another support such as an eyeglass frame remains substantially in place on a wearer, in order to align the plurality of stimuli with a peripheral retina of a wearer. In some embodiments, each projection module comprises one or more of a source of illumination, a stimulus forming element that generates a stimulus when illuminated by the source of illumination, or a guide that directs the generated stimulus to an optical element. The optical element directs the generated stimulus to a desired region or location of a person's eye where the stimulus forms an image that is defocused on the retina. Depending on the type of refractive error being treated, the region or location of the eye may be anterior or posterior to a peripheral region of the retina of the eye.

In some embodiments, the stimulus forming element may comprise a mask, film, or reticle, which is configured to move relative to a substantially fixed support such as an eyeglass frame in order to place the plurality of stimuli on a peripheral retina of a wearer. In some embodiments, the wearer adjustable optical element may comprise one or more of mirrors, partial mirrors, beam splitters, or lenses that direct the stimulus into the eye to form an image of the stimulus at the desired location. The mirrors can be sized and shaped in many ways and may comprise one or more of flat mirrors, curved mirrors, concave mirrors, convex mirrors, spherically shaped mirrors or aspheric mirrors.

In some embodiments, the wearer adjustable projection modules and optical elements may be incorporated into a virtual or augmented reality device, smartphone, handheld device, tablet computer, electronic game console, or similar device. In these embodiments, the projection modules and optical elements may be incorporated into an existing device or provided as a wearer adjustable add-on or peripheral device.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a cross section of a vision apparatus to treat myopia or other refractive error of an eye, in accordance with some embodiments;

FIG. 2 shows a pair of spectacles that incorporate an apparatus for treatment of a refractive error of an eye, in accordance with some embodiments;

FIGS. 3A and 3B show a lens of the spectacles of FIG. 1B in which an apparatus for treatment of a refractive error of an eye has been incorporated, in accordance with some embodiments;

FIGS. 4A and 4B show a projection module that may comprise part of an apparatus for treatment of a refractive error of an eye, in accordance with some embodiments;

FIG. 5 shows a pair of spectacles for treatment of refractive error of an eye, in accordance with some embodiments;

FIG. 6 shows an isometric view of the spectacles of FIG. 5, in accordance with some embodiments;

FIG. 7 shows a bottom isometric view of a portion of the spectacles of FIG. 5, in accordance with some embodiments;

FIGS. 8A and 8B show a pair of spectacles for treatment of refractive error of an eye and an adjustable retainer, respectively, in accordance with some embodiments; and

FIGS. 9A and 9B show spectacles for treatment of refractive error of an eye with various nose pads, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

In some embodiments, the treatment apparatus is wearer adjustable and comprises two oculars, e.g. left and right, which are connected to each other by a mechanism such as a slide, that can be used to move the oculars along a generally nasal-temporal direction. In some embodiments, each ocular is self-contained with all optics, electronics, and a charging connection supported with the ocular, such that the ocular and these components can move relative to a support such as an eyeglass frame.

The presently disclosed methods, systems and apparatuses are well suited with many types of prior light therapy devices, such as those directed to the treatment of myopia and amblyopia. The stimulus may comprise any suitable stimulus such as a light spot or a plurality of light spots. The stimulus may comprise any suitable stimulus such as a spot of light on the retina, and may be focused or defocused on the retina, for example. In some embodiments, the light therapy provides light to increase the blood flow and metabolic rate of the fundus, for example with low levels of light such as near infrared light or red light. The stimulus may also comprise any suitable wavelength, such as one or more of near infrared, red, orange, yellow, blue, indigo, violet, or ultraviolet light, for example. Also, the stimulus may comprise any suitable intensity for the treatment of refractive error, such as low light levels.

In some embodiments, the apparatus is configured for the wearer to adjust the stimulation optics while being worn, in order to improve alignment. In some embodiments, the wearer puts on the glasses, looks into a mirror to adjust the position of the oculars, until both sets of stimuli are situated in the peripheral vision of the left and the right eye. In some embodiments, the slide comprises a pin or a screw to restrain the oculars in place once the adjustment is completed. This can be beneficial because the adjustment can be repeated when the wearer is looking far or near, or at a computer, which improves placement of the stimuli on the peripheral retina with these viewing conditions.

In some embodiments, the stimulation optics are optically coupled to an optical element such as a lens, flat or other optically transmissive structure and arranged with respect to the optical element such that the wearer has a clear central zone of vision. In some embodiments, the optical element comprises a lens configured to correct a refractive error of the eye at least partially. In some embodiments, the lens comprises a sphere, a cylinder and an axis configured to correct refractive error of the eye comprising a sphere, a cylinder, and an axis. Alternatively, the lens may comprise a spherical lens which comprises an optical power corresponding to the spherical equivalent optical power of the eye, i.e. sphere plus cylinder divided by two. For patients with small amounts of astigmatism, a spherical lens corresponding to the spherical equivalent of the eye provides vision that is very close to vision with cylindrical correction and the difference can be essentially imperceptible for at least some people.

The wearer adjustable light sources can be configured in many ways. In some embodiments, the light sources are mechanically coupled to the corrective lens and move with the corrective lens. Alternatively, the wearer adjustable light sources can be configured to move independently from the lens, which can be helpful in some instances.

In some embodiments, each projection module comprises one or more of a source of illumination, a stimulus forming element, or a guide element. The guide element may comprise a light tube or lightguide. The light tube or lightguide may comprise a mirror used to direct the formed stimulus to a lens or lenses. The mirror can be sized and shaped in many ways and may comprise one or more of a flat mirror, a curved mirror, a concave mirror, a convex mirror, a spherically shaped mirror or an aspheric mirror. The mirror may comprise a flat mirror, a partial mirror, or concave mirror, for example. The lens or lenses of the light tube or lightguide direct and focus the stimulus to one or more of a set of optical elements. The optical elements redirect the stimulus to form an image of the stimulus anterior or posterior to a peripheral region of the retina.

The optical elements may comprise a mirror or mirrors, a partial mirror (or set of partial mirrors), a beam splitter or beam splitters, a lens or lenses, or a concave surface or surfaces. These optical elements may be embedded in, applied to, or formed on an optic or contained in a lightguide that is embedded in, applied to, or formed on the optic. The mirror can be sized and shaped in many ways and may comprise one or more of a flat mirror, a curved mirror, a concave mirror, a convex mirror, a spherically shaped mirror or an aspheric mirror. The mirror(s) may be a flat mirror, partial mirror, or a concave mirror, for example. The projection modules may be supported by, or embedded in or partially embedded in, a frame of a pair of spectacles. In these embodiments, the optical elements may be embedded in, formed on, or applied as a film or layer to a lens of the spectacles. A facet in a lens may be used to direct a stimulus provided by a projection module to an optical element. Both the projection modules and the optical elements may be incorporated in or formed on a contact lens. Further, both the projection modules and the optical elements may be incorporated in a film or layer that is applied to the front surface or back surface of a corrective lens.

The projection modules may include circuitry for operating or activating the source of illumination. The source of illumination may be a LED, OLED, a phosphorescent LED or a plurality of LEDs. The circuitry may be a printed circuit board (PCB) that implements logic to control or activate the source of illumination. The projection module may also include a source of power, such as a battery. The logic implemented by the circuitry may be implemented in the form of a processor programmed with a set of executable instructions. The stimulus forming element may comprise a mask, film, or a reticle. The image formed by the projection module and optical element may comprise an illuminated cross on a dark background and optionally a white cross on a black background.

Although specific reference is made to spectacles and contact lenses herein, the disclosed apparatus and methods are suited for use with, or incorporation into, other devices or systems. A person of ordinary skill in the art will readily appreciate how one or more of the disclosed components or elements may be implemented as part of other systems or devices, based on the teachings provided herein.

For example, the projection modules and/or optical elements may be partially or wholly incorporated into one or more of an ophthalmic device, a TV screen, a computer screen, a virtual reality (“VR”) display, an augmented reality (“AR”) display, a handheld device, a mobile computing device, a tablet computing device, a smartphone, a wearable device, a spectacle lens frame, a spectacle lens, a near eye display, a head-mounted display, a goggle, a contact lens, an implantable device, a corneal onlay, a corneal inlay, a corneal prosthesis, or an intraocular lens.

Although the presently disclosed methods and apparatus can be used to treat many types of refractive error, the presently disclosed methods and apparatus are well suited to treat the progression of myopia, for example.

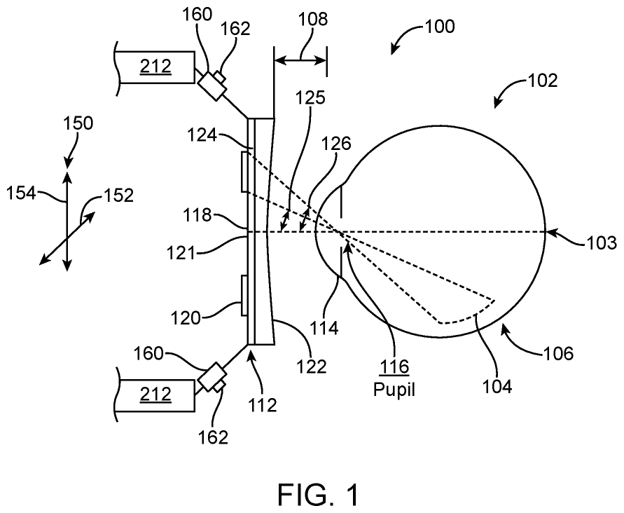

FIG. 1 shows a cross section of a vision apparatus 100 to treat myopia or other refractive error of an eye 102, in which the placement of apparatus 100 is wearer adjustable in relation to a wearable support 212, such as a frame, with a wearer adjustable retainer 160, such as one or more of a slide, a pivot or other structure as described herein. In some embodiments, the wearer is able to adjust placement of the apparatus 100 while viewing an object in order to provide one or more stimuli on a desired location of the retina such as the peripheral retina as described herein. In some embodiments, the wearer adjustable retainer 160 comprises a releasable retainer 162, which allows the wearer to move the stimulus projection components with respect to the wearable support 212 when released, and to fix the stimulus projection components in place with respect to the support when engaged.

While the apparatus 100 can be configured in many ways, in some embodiments the apparatus 100 is configured to allow the user to adjust the placement of projection optics while viewing one or more stimuli in order to decrease interference from a pupil of the eye, which could potentially decrease the visibility of the one or more stimuli. In some embodiments, improper alignment of the one or more stimuli can result in the one or more stimuli appearing dimmer, truncated or not visible, for example.

The adjustable retainer 160 can be configured in many ways to allow movement 150 of the vision apparatus 100 in relation to wearable support 212. In some embodiments, movement 150 comprises movement in a nasal temporal direction 152, or in a superior inferior direction 154, and combinations thereof. In some embodiments, the adjustable retainer is coupled to the plurality of projection modules to allow the wearer to move the plurality of projection modules relative to the wearable support to position the stimulus at a location of a retina of the eye of the wearer to decrease the progression.

The projection modules and optical elements described herein may be implemented as part of the optic 112 by use of any suitable process. Such processes include embedding, etching, film deposition, photo-lithography, insertion into a cavity, molding, etc. The images generated by the stimulus forming element described herein are transmitted through the optic 112 to the eye 102 of the wearer, represented by the cornea 114 and the pupil 116. The optic 112 may comprise a refractive lens that changes the focus of the light before the light enters the eye 102 of a wearer. The optic may instead comprise a film or layer that is applied to a lens, either to the front or back of a lens.

The optic 112 may include a posterior optical structure 122 that may be curved or otherwise shaped to adjust the focus of the stimuli onto the wearer's eye 102. For example, in some embodiments in which, for example, the projection modules and optical elements are implemented as part of, or as an addition to spectacles, the optic 112 may comprise a prescription lens to correct refractive errors of the patient's eye 102 with the posterior optical surface 122 shaped to correct one or more of myopia, hyperopia, astigmatism, and other refractive errors of the eye 102.

A defocus treatment device 124 may be attached to, embedded in, or formed as part of a surface of, the optic 112. For example, in FIG. 1 the defocus treatment device 124 is a part of, or attached to, the front surface of the optic 112. In some embodiments, the treatment device 124 is adhered to the optic 112 with an adhesive, such that the treatment device 124 and optic 112 move together when the wearer adjusts mechanism 160. Alternatively, the optic 112 may not be attached to treatment device 124 and may remain substantially fixed with respect to the support 212 when the treatment device 124 moves with respect to the support 212 in order to place the treatment stimuli at the desired location of the retina. In some embodiments, the defocus treatment device 124 comprises a peripheral defocus optical structure 120 arranged around a central optical zone 118, in order to provide clear central vision to the wearer. In some embodiments, the components of the defocus structure 120 are generally arranged about a center 121, and the defocus structure 120 and center 121 are coupled to the support 212 with the wearer adjustable retainer so as to allow the wearer to adjust the defocus structure 120 and corresponding center 121, to allow the wearer to adjust the position of the stimuli on the retina. In some embodiments, the defocus structure 120 comprises a light focusing element, the light focusing element comprising one or more of a lens, a spherical surface, a grin lens, a diffractive optical element, a holographic optical element, or a curved mirror. In some embodiments, the defocus optical structure 120 alters the focus of the light. The defocus optical structure can be configured to form a stimulus image 104 anterior to the retina 106 to treat refractive error of the eye 102 such as myopia. Alternatively, the defocus optical structure 120 can be configured to form an image of the stimulus 104 posterior to the retina 106 of the eye 102.

Although in some embodiments, reference is made to the treatment defocus device 120 being adhered to an optic 112 where the optic is a lens, in some embodiments the defocus optical structure 120 is formed directly on the surface of a lens, for example with structures etched into the surface of a lens. In some embodiments, the defocus optical structure 120 is formed as a module that is embedded in a lens or is formed as part of a process of molding a lens.

The dimensions of the optical zone 118 and peripheral defocus optical structure 120 zone can be configured in many ways. In some embodiments, the optical zone 118 is configured to provide a clear view of an object on the macula 103 while the peripheral defocus structure 120 provides the stimulus to the peripheral retina. In some embodiments, the peripheral defocus optical structure 120 is sized and shaped to transmit light at an angle within a range from 12 degrees to 40 degrees with reference to an entrance pupil of the eye 102 or within a range from 15 to 35 degrees, for example. In some embodiments, the angle comprises a half-angle, such as an angle between the boundary of the optical zone and a line formed through the center of the optical zone and the center of the entrance pupil. In some embodiments, the peripheral defocus optical structure 120 is sized to be at an angle within range from 15 degrees to 50 degrees with reference to an entrance pupil of the eye, for example.

In some embodiments, the peripheral defocus optical structure 120 comprises an inner boundary and an outer boundary. The inner boundary corresponds to an inner boundary angle 125 within a range from 15 degrees to 20 degrees with reference to the entrance pupil 116 of the eye 102 and the outer boundary corresponds to an outer boundary angle 126 within a range from 25 degrees to 70 degrees with reference to the entrance pupil of the eye 102. In some embodiments, the lens is a distance 108 from the eye 102. The distance, the inner boundary, and the outer boundary may be dimensioned to provide the inner angle and the outer angle with reference to the entrance pupil of the eye 102.

The peripheral defocus optical structure 120 may be annular in shape, having an inner diameter and an outer diameter selected such that the peripheral defocus is applied to a portion of the retina of the patient's eye 102 that is eccentric to the fovea. For example, the inner diameter may be at an angle of about 7.5 degrees with respect to an optical axis of the optic 112 and pupil, this angle may be referred to as an inner boundary angle 125. The outer diameter of the peripheral defocus optical structure 120 may be at an outer boundary angle 126 with respect to the optical axis of the primary eye 102 and the people, for example at 17.5 degrees. Such an arrangement, results in the peripheral defocus optical structure 120 being located in a peripheral field of view of the wearer with a corresponding defocus of the projected light in a peripheral region of the wearer's retina eccentric to the fovea.

Although reference is made to an annular shape, the peripheral defocus optical structure 120 can be configured with other shapes, such as polygons, squares, triangles, and may comprise a plurality of discrete optical structures located around the optical zone at appropriate locations.

In some embodiments, the peripheral defocus optical structure 120 may include optics or optical structures that change the focus of the light projected into a person's eye 102. Peripheral defocus optical structure 120 may comprise one or more of diffractive optics, lenslets, gradient index (“GRIN”) lenslets, crossed cylindrical rods, masks, or echelettes that alter the focus of light passing through the defocus optical structure 120.

In some embodiments, the peripheral defocus optical structure 120 is dimensioned to provide defocused images to a peripheral portion of the retina. In some embodiments, the defocus optical structure 120 is configured to provide a stimulus to a peripheral portion of the retina that comprises a region of the retina outside the fovea or the macula 103, so as to provide clear vision to the fovea and the macula 103 when the wearer looks ahead and the peripheral defocus optical structure 120 provides a defocused image onto the peripheral retina. The image may be defocused in a range between 2.0 to 6.0 Diopters (“D”) myopically or hyperopically with respect to the retina. For example, the defocus may be 3.5 D to 5 D anterior to the retina, e.g. myopic defocus, or posterior to the retina, e.g. hyperopic defocus. The defocus is preferably between 2.5 D to 5.0 D, and more preferably between 3.0 D to 5.0 D.

In some embodiments, a defocus treatment device includes use of localized stimuli projected into the peripheral zone to treat refractive errors of the eye 102. In the defocus treatment device 124, the stimuli are projected through the peripheral defocus optical structure 120 and accordingly, the stimuli are defocused by the peripheral defocus optical structure.

For the treatment of spherical refractive errors of the eye 102, such as myopia, the stimulation projected to the retina 106 may be uniform about the periphery of the central optical zone 118. For the treatment of cylindrical refractive errors of the eye 102, such as astigmatism, the stimulation projected to the retina may be non-uniform about the periphery of the central optical zone 118. For example, the stimulation may be greater along a meridian corresponding to or aligned with an astigmatic first axis of the eye 102 and symmetrically mirrored about a second astigmatic axis of the eye 102.

The adjustable retainer 160 can be configured in many ways to provide movement 150 as described herein. In some embodiments, the adjustable retainer is configured to move the plurality of projection modules relative to the wearable support while the optic remains substantially fixed relative to the wearable support. Alternatively or in combination, the adjustable retainer is coupled to the optic and configured to move the optic and the plurality of projection modules together relative to the wearable support. The adjustable retainer 160 may comprise one or more of a slide, a rail, a slot, a pivot, a hinge, a living hinge, an extension and a guide to receive the extension, a telescopic mechanism, or a rack and pinion, for example. In some embodiments, the adjustable retainer is configured to fix a position of the plurality of projection modules relative to the wearable support. The retainer may comprise one or more of a screw, a thumb screw, a friction pad, a brake, a hole, an extension, a guide to receive an extension, or a detent. The retainer can be configured in many ways, for example with a suitable amount of friction to allow the wearer to adjust the placement of the projection modules and to retain the projection modules in position once placed by the wearer.

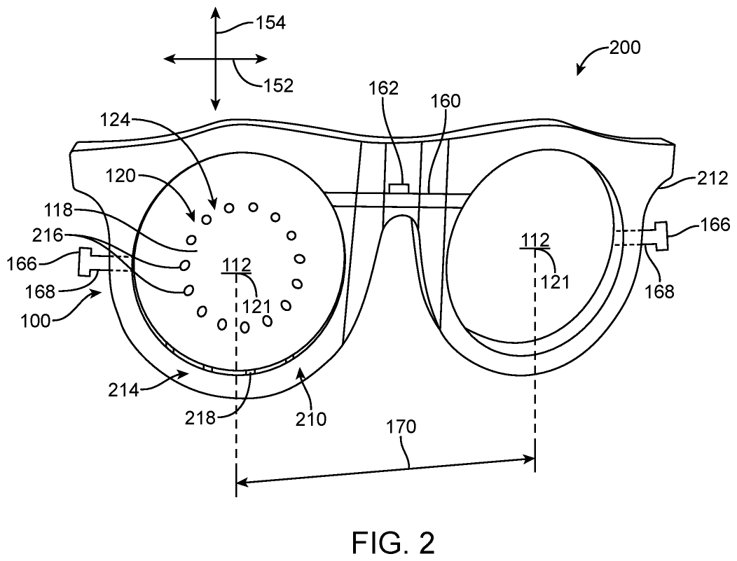

FIG. 2 shows a pair of wearer adjustable spectacles 200 comprising apparatus 100 and wearer adjustable retainer 160 for treatment of a refractive error of an eye, in accordance with some embodiments. In some embodiments, the apparatus 100 comprises a plurality of projection modules, each with its own optics 210, that are arrayed circumferentially around the central optical zone of a lens or other type of optic 112 and within the support 212, such as frame of a pair of spectacles 200. Each of the projection modules 214 comprises a light source with a mask or other element that is illuminated with the source to provide the stimulus. The wearer adjustable retainer 160 is configured to allow the wearer to adjust a separation distance 170 between a first center 121 of first projection modules and a second center 121 of second projection modules. The adjustable retainer 160 may comprise one or more engagement structures 166 that allow the wearer to adjust distance 170 with movement along nasal temporal direction 152, for example. The wearer adjustable retainer 160 comprising retention element 162 allows the wearer to adjust distance 170, for example by pressing inward on engagement structures 166 toward each other for near vision, or by pulling engagement structures 166 away from each other for far vision. The one or more engagement structures 166 may comprise one or more extensions 168, which are coupled to the projection optics, e.g. optics of the projection modules, to allow the wearer to adjust the positions of the stimuli projected onto the wearer's retinas. The projection optics can be coupled to the adjustable retainer so as to move independently of the one or more optics 112, or to move with the one or more optics 112 such as when the projection optics are affixed to the one or more optics. In some embodiments, the wearer adjustable retainer 160 is configured with a suitable amount of friction, so as to allow the wearer to adjust distance 170 and to allow distance 170 to remain substantially fixed while the wearer performs other activities, such as reading or athletic activities such as jogging.

The light source may comprise an LED, OLED, or other form of display. The projection module 214 may include a light tube or lightguide (termed a “guide element” herein) that directs the stimulus to an optical element. The light tube or lightguide may include a mirror which directs the formed stimulus to a set of optical elements 216 (for example, mirrors, partial mirrors, beam splitters, or lightguides) that are arranged on or in a lens 112. The optical elements 216 redirect the stimulus or image through the spectacle lens to the eye. As shown in FIG. 2, in some embodiments, a facet or facets 218 in a lens are used to transmit a stimulus or image from a projection module that is incorporated into a spectacle frame to a set of optical elements.

In some embodiments, the mirrors are configured to reflect a narrow bandwidth of light and to transmit light at other wavelengths. The reflective bandwidth can be within a range from about 5 nm to about 50 nm (or 10 nm to 25 nm) for the full width half maximum, for example, in order to transmit wavelengths outside the reflective bandwidth. This approach can provide a mirror that is substantially transparent and allow the optic, e.g. lens, to transmit light with a refraction that is substantial similarly to regions of the optic without the mirrors, e.g. the optical zone. This approach can allow the wearer to have substantially clear focused vision in the regions with the mirrors. When the projection modules are activated, the stimuli are imaged anterior or posterior to the retina and perceptible to the wearer. With these embodiments, the light source typically comprises a bandwidth that is typically less than the reflective bandwidth to decrease stray light, although the light source bandwidth may be greater than the bandwidth of the mirrors. The bandwidth of the plurality of light sources can be within a range from 5 nm to 50 nm, for example from 10 nm to 25 nm, for the full width half maximum of the bandwidth of the light source. In some embodiments, the mirrors comprise dichroic mirrors with one or more layers to provide an appropriate bandwidth.

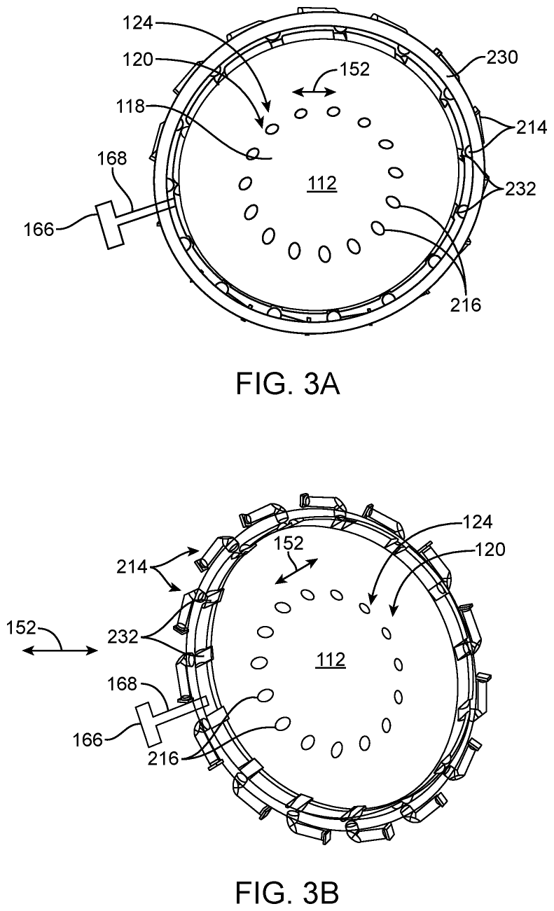

FIGS. 3A and 3B show a lens 112 of the spectacles 200 of FIG. 2 in which an apparatus 100 for treatment of a refractive error of an eye has been incorporated, in accordance with some embodiments. As shown in the figures, arranged around a periphery of the lens are a plurality of projection modules 214, where in some embodiments the projection modules may be fabricated as elements of a printed circuit board (PCB) 230. The projection modules 214 generate a stimulus that is provided to a facet 232 in the lens 112. The facet 232 acts to direct the generated stimulus to an embedded or applied optical element 216, such as a mirror, partial mirror, beam splitter, or lightguide. The optical elements 216 of FIGS. 3A and 3B may comprise imbedded mirrors.

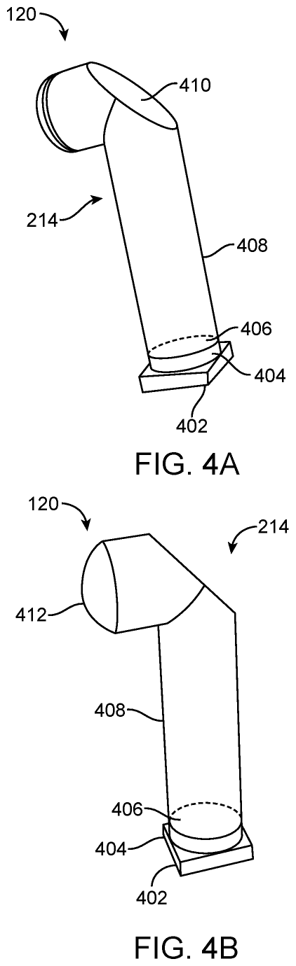

FIGS. 4A and 4B show a projection module 214 that may be part of an apparatus 100 for treatment of a refractive error of an eye, in accordance with some embodiments. As shown in FIGS. 4A and 4B, some embodiments of a projection module include a PCB 402 (which may contain or implement circuitry, a processor, or control logic), a source of illumination 404, an optical structure 406 such as a mask, film, or reticle that is used to generate or form a stimulus, and a light tube or lightguide that transmits the generated stimulus to a mirror 410. In this embodiment, the mirror 410 acts to redirect the generated stimulus to a lens or lenses 412. The lens or lenses 412 of the light tube or lightguide act to direct and focus the generated stimulus to one or more optical elements 216, such as the mirrors described with reference to FIGS. 1B, 2, 3A, and 3B.

The light tube or lightguide 408 may contain a convex surface 412, a lens, or lenses that create convergent light exiting the light tube or lightguide 408. As mentioned, in some embodiments, a facet on the edge of the spectacle lens or other lens directs the light from the projection module 214 to an optical element 216 which is embedded in, formed on, or applied to the lens. The optical element (for example, a mirror) directs the light such that it focuses an image at the appropriate location in the eye.

Additional examples of light sources and projection optics suitable for incorporation with the wearer adjustable retainer 160 to enable wearer alignment of the projected stimuli as described herein are described in U.S. patent application Ser. No. 16/947,537, filed Aug. 5, 2020, entitled “DEVICE FOR PROJECTING IMAGES ON THE RETINA”, now U.S. Pat. No. 11,358,001, issued Jun. 14, 2022, U.S. patent application Ser. No. 17/304,691, filed Jun. 24, 2021, entitled “PROJECTION OF DEFOCUSED IMAGES ON THE PERIPHERAL RETINA TO TREAT REFRACTIVE ERROR”, now U.S. Pat. No. 11,275,259, issued Mar. 15, 2022, and U.S. patent application Ser. No. 17/303,889, filed Jun. 9, 2021, entitled “APPARATUS AND METHODS FOR THE TREATMENT OF REFRACTIVE ERROR USING ACTIVE STIMULATION”, now U.S. Pat. No. 11,281,022, issued Mar. 22, 2022, which have been previously incorporated by reference.

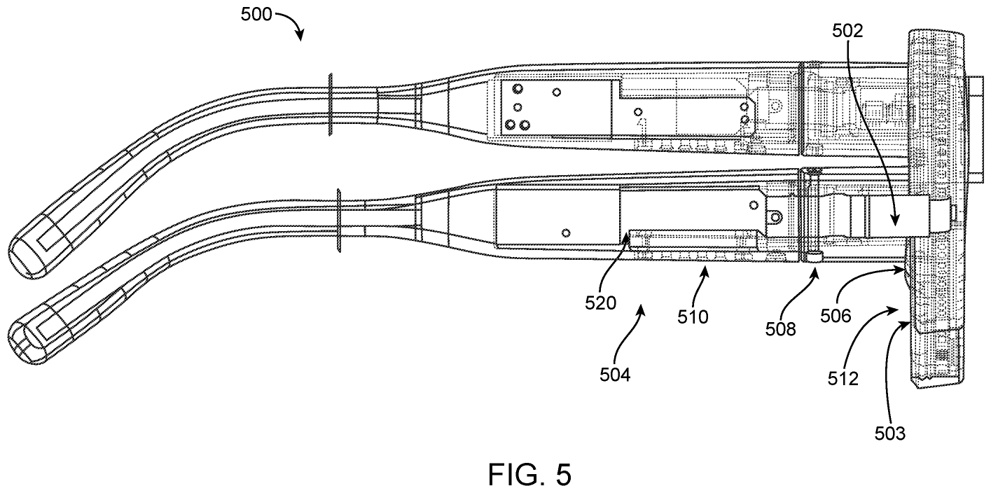

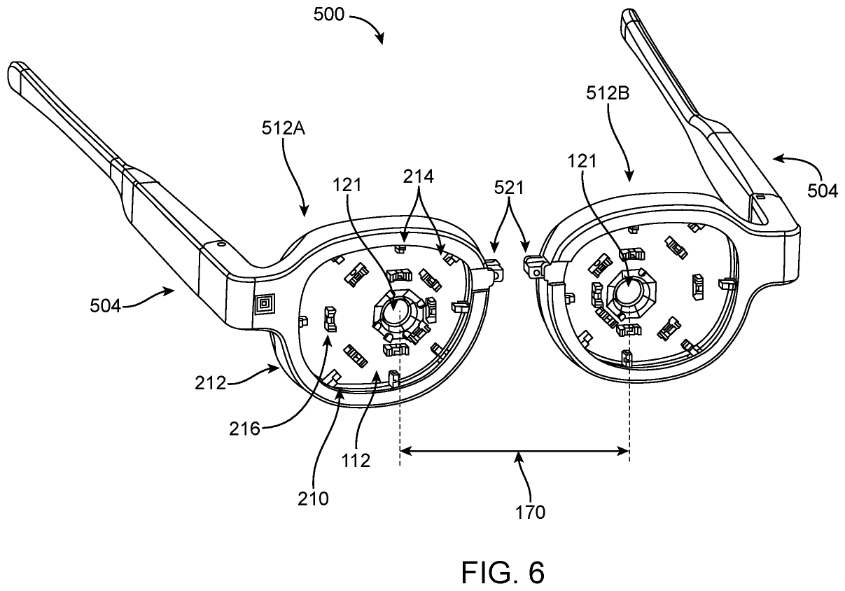

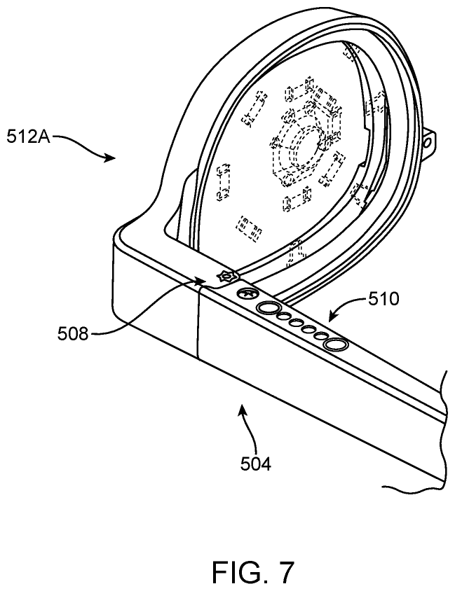

With reference to FIGS. 5, 6, and 7 a pair of spectacles for treatment of refractive error of an eye, in accordance with some embodiments, is depicted. FIG. 5 depicts a pair of adjustable electronic spectacles 500 configured to project a defocused image on the retina away from the central field that includes the fovea for treatment of a refractive error of an eye. The adjustable electronic spectacles 500 may include a pair of lenses 112 that may be corrective lenses to correct for refractive errors of a patient's eye. The lens 112 may also include optical and electrical components 503 for the treatment of refractive errors of the patient's eyes. The components 503 may include one or more light injectors, lenses, or mirrors for the controlled projection of images onto the patient's retina, as described herein.

In some embodiments, the components 503 comprise a plurality of projection modules, each with its own optics, such as optics 210, that are arrayed circumferentially around the central optical zone of a lens or other type of optic 112 and within the support 512, such as frame of a pair of spectacles 500. Each of the projection modules 214 comprises a light source with a mask or other element that is illuminated with the source to provide the stimulus.

The light source may comprise an LED, OLED, or other form of display. The projection module 214 may include a light tube or lightguide (termed a “guide element” herein) that directs the stimulus to an optical element. The light tube or lightguide may include a mirror which directs the formed stimulus to a set of optical elements 216 (for example, mirrors, partial mirrors, beam splitters, or lightguides) that are arranged on or in a lens 112. The optical elements 216 redirect the stimulus or image through the spectacle lens to the eye. In some embodiments, a facet or facets 218 in a lens are used to transmit a stimulus or image from a projection module that is incorporated into a spectacle frame to a set of optical elements.

In some embodiments, the mirrors are configured to reflect a narrow bandwidth of light and to transmit light at other wavelengths. As discussed herein, the reflective bandwidth can be within a range from about 5 nm to about 50 nm (or 10 nm to 25 nm) for the full width half maximum, for example, in order to transmit wavelengths outside the reflective bandwidth. This approach can provide a mirror that is substantially transparent and allow the optic, e.g. lens, to transmit light with a refraction that is substantial similarly to regions of the optic without the mirrors, e.g. the optical zone. This approach can allow the wearer to have substantially clear focused vision in the regions with the mirrors. When the projection modules are activated, the stimuli are imaged anterior or posterior to the retina and perceptible to the wearer. With these embodiments, the light source typically comprises a bandwidth that is typically less than the reflective bandwidth to decrease stray light, although the light source bandwidth may be greater than the bandwidth of the mirrors. The bandwidth of the plurality of light sources can be within a range from 5 nm to 50 nm, for example from 10 nm to 25 nm, for the full width half maximum of the bandwidth of the light source. In some embodiments, the mirrors comprise dichroic mirrors with one or more layers to provide an appropriate bandwidth.

As shown in the figures, arranged around a periphery of the lens are a plurality of projection modules 214, where in some embodiments the projection modules may be fabricated as elements of a printed circuit board. The projection modules 214 generate a stimulus that is provided to a facet in the lens 112. The facet acts to direct the generated stimulus to an embedded or applied optical element 216, such as a mirror, partial mirror, beam splitter, or lightguide.

In some embodiments, for the treatment of spherical refractive errors of the eye, the plurality of light sources, such as projection units, are arranged with symmetrically with respect to central axis of the spectacle lens, the center of the spectacle lens, or another location of the spectacle lens. The symmetry may be rotational symmetry, such that the light sources are arranged on a circumference centered on the location of the spectacle lens.

In some embodiments, for example, for the treatment of astigmatism, the adjustable electronic spectacle 500 is configured to project defocused images with respect to the astigmatic axes of the patient's eye to provide different amounts of stimulation to different regions of the peripheral retina. In some embodiments, the light sources such as projection units are located along the astigmatic axis, although the light sources may be located at other locations. The light sources can be configured to provide different amounts of stimulation to the peripheral retina in accordance with the refractive error of the eye. In some embodiments, the light sources are configured to provide different amounts of illumination along different axes in order to promote different changes in choroidal and scleral tissue corresponding to different changes in axial length as described herein. The spectacle lens may comprise an optical zone configured to correct astigmatic refractive errors in accordance with the first axis and the second axis.

In some embodiments, the spectacle lenses 112 may include an optical zone that can be appropriately sized for the pupil of the eye and the illumination conditions during treatment. The central optical zone is designed to provide emmetropic correction or other suitable correction to the wearer and may be provided with both spherical and astigmatic correction. The central optical zone is circumscribed by an outer annular zone, such as a peripheral zone. The optical zone is configured to provide refractive correction and can be spherical, toric or multifocal in design, for example with a visual acuity of 20/20 or better.

The spectacle lenses 112 includes a plurality of embedded projection units. Each of the plurality of projection units comprises a light source and one or more optics to focus light in front of the retina as described herein. Each of the optics may comprise one or more of a mirror, a plurality of mirrors, a lens, a plurality of lenses, a diffractive optic, a Fresnel lens, a light pipe, an optical fiber or a wave guide. The spectacle lenses 112 may be sphero-cylindrical equivalent lenses. The lenses may be molded lenses. In some embodiments, the first support 512A and the second support 512B may be assembled with the molded sphero-cylindrical lenses.

The spectacles 500 may include two temple portions 504, each of which comprises an elongate extension configured to engage a temple of the subject and may extend toward a pinna of the ear for support with the pinna. The temple portion of the spectacles may be connected via a hinge 508 to an end piece 506. The end piece 506 couples the hinge to the frame of the lenses 112. The end piece 506 may include a cavity 502 for housing electrical and control system of the adjustable electronic spectacles 500. The temple portion 504 may include a cavity 520 for housing electrical and control system of the adjustable electronic spectacles 500. An electrical connection 510 may be included in the spectacles 500. In some embodiments, the electrical connection 510 may be located on a temple portion 504 of the spectacles 500. In some embodiments, the electrical connection 510 may be located on a bottom side of a temple portion 504 of the spectacles 500. In some embodiments, the electrical connection 510 may be located at the component cavity 520.

The spectacles component cavity 520 in the temple portion 504 and/or the cavity 502 in the temple portion 504 may comprise a battery, microcontroller, processor, memory, including non-transitory memory, and communication circuits, such as wired or wireless communication circuitry and one or more antennae for electronic communication. Although reference is made to a battery, the spectacle may comprise any suitable energy storage device.

The projection modules 214 can be configured to provide defocused images to the peripheral portion of the retina as described herein and may include light sources and projection optics. In some embodiments, one or more projection optics are configured with the light sources to project a defocused image from the light sources onto the peripheral retina away from the central visual field that includes the macula in order to stimulate a change in choroidal thickness, such as an increase or decrease in choroidal thickness. In some embodiments, changes in choroidal thickness comprise markers that are related to efficacy and can be used as a preliminary determination of efficacy. In some embodiments, efficacy comprises a stabilization of axial length and refractive error in response to treatment as described herein.

The one or more projection units can be configured to stimulate the retina without degrading central vision and corresponding images formed on one or more of the foveal or macular regions of the retina. In some embodiments, the one or more projection optics do not decrease the image forming characteristics of the vision correction optics prescribed to correct refractive errors of the wearers. This configuration can allow the wearer to have good visual acuity while receiving therapy from the defocused images as described herein.

In some embodiments, the light from light sources of the projection units is columnated and focused by one or more projection optics, as described herein. The function of the light sources and the projection optics is to substantially collimate the light emitted by the light sources and focus it at a focus that is designed to be in the front of or behind the retina to provide appropriate defocus to stimulate a change in choroidal thickness.

In accordance with some embodiments, a spectacle 500 comprises projection units which include projection optics and micro-displays as the light source. The micro-displays may comprise an OLED (organic light emitting diode) or an array of micro-LEDs. Light emitted by these displays may be Lambertian. In some embodiments, the micro-display is optically coupled to a micro-optical array that substantially collimates and focuses the light emanating from the micro-display. The micro-display may comprise one or more miniaturized pixels. In some embodiments, the micro-display forms an extended array of pixels, characterized by a pixel size and a pixel pitch, in which the pixel size and the pixel pitch together correspond to a fill factor of the micro-display. In some embodiments, the pixel array is optically coupled with a micro-optic array in order to substantially collimate and focus light from the pixels.

The images created by these displays may be defocused and may be placed symmetrically in four quadrants of the field of view or of the eye (e.g. nasal-inferior, nasal-superior, temporal-inferior and temporal-superior). The micro displays can be located away from the optical center of the lens. The central optic of the lens can be selected to bring the wearer to emmetropia.

The micro-display can be coupled to and supported with the body of the correction optic such as the spectacle lens, for example. In some embodiments, the micro-displays and the micro-optic arrays are mounted immediately adjacent to each other on the same correction optic, separated by a fixed distance in order to project a bundle of rays to the pupil of the eye, at an orientation such that it forms a defocused image at a desired location on the retina as described herein. In some embodiments, the one or more projection optics are mounted on or in the one or more correction optics, such that rays from the projection optics are refracted through the correction optics. The correction optics refract the rays from the projection optics to be convergent or divergent as helpful for clear vision, so that the micro-optical array can provide the desired magnitude of additional power that may be plus or minus, depending on the magnitude and sign of the defocus desired. The micro-display may be monochromatic or polychromatic, for example.

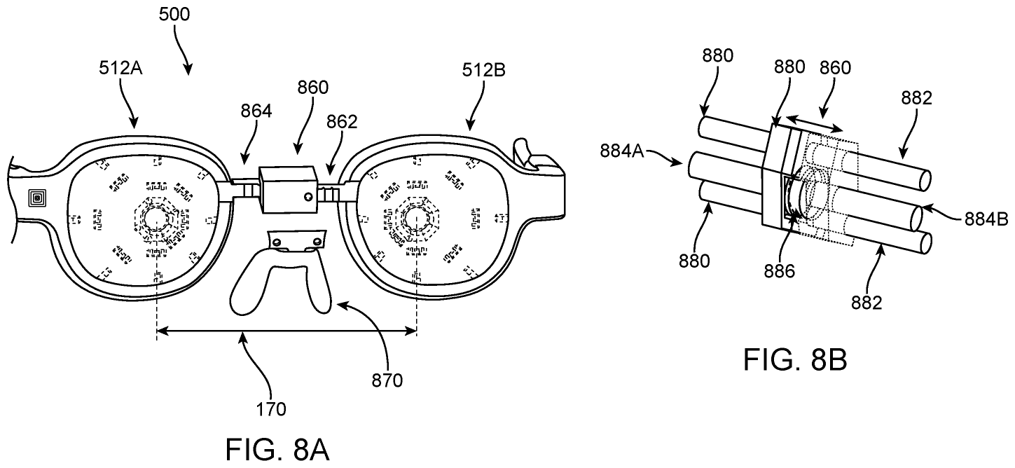

The pair of adjustable electronic spectacles 500 may include a first support 512A adjustably coupled to a second support 512B. A wearer adjustable retainer, such as wearer adjustable retainer 860, see FIGS. 8A and 8B, may be configured to allow the wearer to adjust a separation distance 170 between a first center 121 of first projection modules and a second center 121 of second projection modules.

A first end of the wearer adjustable retainer may be coupled to a first coupling 521 on the first support 512A and to a second coupling 521 on the second support 512B to adjustably join the first support 512A to the second support 512B.

Each of the supports 512A and 512B may have independent and/or separated electrical components 503 in their respective temple portions 504 and end pieces 506 such that the components 503 of support 512A control and power the right side of the spectacles 500 and the components 503 of the support 512B control and power the left side of the spectacles 500. In some embodiments, there are no electrical connections, such as a PCB or wiring through the bridge, between the right and left sides of the spectacles and their associated supports 512A and 512B.

FIGS. 8A and 8B show a pair of spectacles for treatment of refractive error of an eye and an adjustable retainer 860, respectively, in accordance with some embodiments. In some embodiments, the adjustable retainer 860 is similar to the adjustable retainers discussed herein, such as adjustable retainer 160. In some embodiments, the adjustable retainer 860 may be an adjustable bridge. The adjustable bridge may include a user adjustable or wearer adjustable mechanism that is configured to allow the wearer to adjust the distance between the first portion 512A and the second portion 512B. The adjustable bridge 860 may be adjustable coupled to the first portion 512A and the second portion 512B by first and second extensions 862, 864. A user or wearer may adjust the distance between the first and second portions 512A, 512B by extending or retracting the extensions. Adjusting the distance between the first and second portions 512A, 512B may result in or may simultaneously adjust the separation distance 170 between a first center 121 of first projection modules in the first portion 512A and a second center 121 of second projection modules in the second portion 512B.

In some embodiments, the first extension is adjustable independently of the second extension. In some embodiments, the distance from the center 121 of the right eye to the centerline of a patient's face or nose is adjustable separately from the distance from the center 121 of the left eye to the centerline of a patient's face or nose. Separate adjustability allows for configuring the spectacles for people with asymmetrical faces and eye positions.

In some embodiments, nose pads 870 may be attached or otherwise coupled or coupled directly to the adjustable bridge 860.

FIG. 8B shows an embodiment of an adjustable bridge 860. The adjustable bridge 860 may include a housing 880 having guides 882 extending therefrom and including left and right taps 884A, 884B and an adjustment mechanism 886. In some embodiments, the adjustable bridge 860 may be configured such that movement, such as rotation, of the adjustment mechanism 886 causes an extension, such as right tap 884B to extend or retract with respect to the housing 880, thereby causing an attached portion, and its corresponding center, to move. In some embodiments, the adjustable bridge 860 may include two adjustment mechanisms 886 each configured to extend or retract an extension. In some embodiments, the adjustable bridge 860 may include a single adjustment mechanism 886 that is configured to extend or retract both or all of the extensions. In some embodiments, the adjustable mechanism is a threated structure, such as a nut. In some embodiments, the adjustable mechanism is captured or retained by the housing and is rotatable with respect to the housing. Rotating the threaded structure or nut may cause an extension, such as a tap or threaded rod to move in a nasal-temporal direction.

In some embodiments, one or more guides 882 may extend from the housing. The guides 882 may provide stability and/or rigidity. They may extend between the housing 880 and a respective one or both of the supports 512. In some embodiments, the guides 882 are slidingly engaged with the housing 880 such that they do not resist or do not substantially resist nasal-temporal movement.

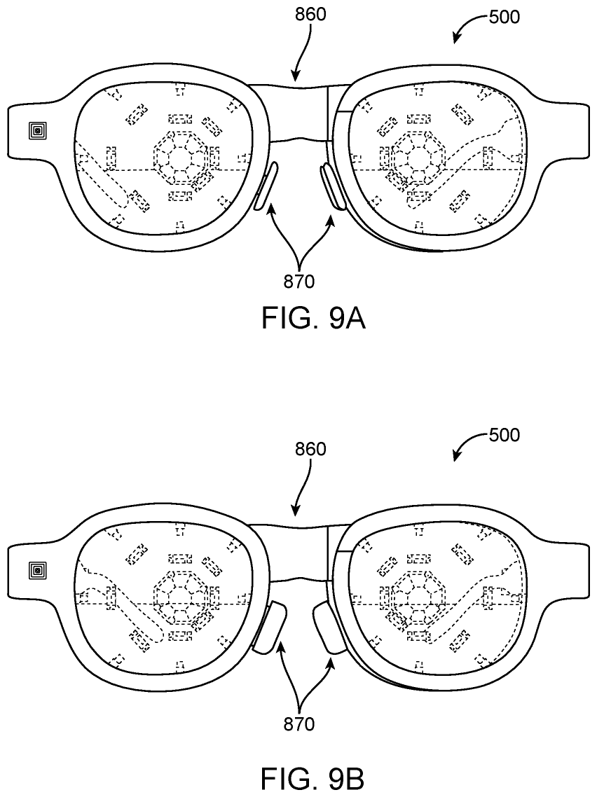

FIGS. 9A and 9B show spectacles 500 for treatment of an eye with various nose pads 870, in accordance with some embodiments. While the nose pads can be configured in many ways, in some embodiments the nose pads comprise variably sized nose pads to allow the location of the optics to be adjusted vertically so as to allow the light beams from the projection optics to pass through the pupil and form the one or more stimuli on the retina with decreased interference from the iris. In some embodiments, an asymmetric pad thickness between a first pad on a first side of the wearer's nose and a second pad on a second side of the wearer's nose can change the location of the projected optics laterally to compensate for asymmetric faces, for example.

While the nose pads 870 depicted in FIG. 8A are coupled to the nose bridge, in some embodiments, the nose pads may be coupled to or directly coupled to the lens frame of the spectacles 500, such as shown in FIGS. 9A and 9B. In some embodiments, a pair of spectacles may be supplied with pairs of different sized nose pads 870 so that a user or wearer can select and apply an appropriately sized pair of nose pads, based for example, on their nose size and interpupillary distance and frame size. The nose pads 870 of FIG. 9A may be 2 mm in thickness while the nose pads 870 of FIG. 9B may be 4 mm in thickness. In some embodiments, pairs of nose pads having thicknesses of 1 mm, 2 mm, 3 mm, and 4 mm may be included with the spectacles. In some embodiments, pairs of nose pads having thicknesses between 1 mm and 6 mm may be included with the spectacles. The nose pads of different thickness can be used to adjust the projection optics inferiorly or superiorly relative to the eye while maintaining the inter pupillary distance (“IPD”), for example.

Adjusting the fit of the spectacles and centers of the lenses may include adjusting the position along the nasal-temporal axis 152, sometime referred to as the nasal-temporal distance between the optical centers of the left and right optics. Such adjustment may include matching the centers of the optic with the interpupillary distance of the patient or otherwise aligning the optical centers of the lenses with the center of the pupils of the respective eyes at a particular vergence.

Adjusting the fit of the spectacles may include adjusting the frames up and down, such as in a superior inferior direction 154. The spectacles may be adjusted such that the center of each respective lens aligns in a superior inferior direction with the center of the patient's pupils by adjusting the nose pads. For example, in the embodiment shown in FIGS. 9A and 9B, the location of the nose pads on the lens frame may be adjusted up and down to adjust the spectacles in a superior inferior direction. In some embodiments, the nose pads may include a wire attachment that attaches the nose pads to the spectacles. The shape of the wire attachment may be adjusted to adjust the superior inferior position of the spectacles.

In some embodiments, the ends of the temple portions of the spectacles are adjusted to couple the nose pads more stably to the nose, e.g. anchor the nose pads to the nose, for example by reshaping the ends of the temple portions to decrease slippage between the nose pads and the nose. While the vertex distance can be adjusted in many ways as described herein, in some embodiments, the vertex distance of the spectacles may be adjusted by reshaping, such as bending the ends of the temple portions so that the engagement of the proximal end of the left and right temple portions with the left and right sides of the head of the wearer is shifted in order to adjust the vertex distance.

The adjustable retainer, nose pads and temple portions can be configured in any suitable way to provide improved alignment. In some embodiments, the adjustable nose bridge is configured for the wearer to adjust the first support and the second support in a nasal-temporal direction, e.g. along an X direction. For example, a screw in the adjustable nose bridge can be turned until the distance between the optical centers of the left and right eye optics are aligned to the center of the pupils of the left and right eye for a particular vergence, i.e. viewing distance. In some embodiments, the one or more adjustable pads is configured for the wearer to adjust the plurality of projection optics in an inferior-superior direction with respect to one or more eyes of the wearer, e.g. along a Y direction. The one or more adjustable pads may comprise a first pad to engage a first side of a nose of the wearer and a second pad to engage a second side of the nose, for example with a bendable structure. In some embodiments, one or more adjustable temple portions are configured for the wearer to adjust a vertex distance between the plurality of projection modules and the eyes of the wearer, e.g. along a Z direction. The one or more temple portions may comprise a first temple portion to engage a first temple and a second temple portion to engage a second temple opposite the first temple.

The present disclosure includes the following numbered clauses.

Clause 1. An apparatus to decrease a progression of a refractive error of eyes of a wearer, comprising: a first support and a second support, the first and second support each comprising: an optic, and a plurality of projection modules coupled to the optic, each of the plurality of projection modules operating to generate and direct light to form a stimulus; and an adjustable bridge that couples the first support to the second support, the adjustable bridge configured to allow the wearer to move the plurality of projection modules of the first support relative to the projection modules of the second support to position the stimulus of the first support and the stimulus of the second support at respective locations of respective retinas of the eyes of the wearer to decrease the progression.

Clause 2. The apparatus of the preceding clause, wherein the adjustable bridge is configured to move the first support relative to a centerline of the apparatus.

Clause 3. The apparatus of any one of the preceding clauses, wherein the adjustable bridge is configured to move the second support relative to the centerline of the apparatus.

Clause 4. The apparatus of any one of the preceding clauses, wherein the adjustable bridge is configured to independently move the first and second supports relative to the centerline of the apparatus.

Clause 5. The apparatus of any one of the preceding clauses, wherein the adjustable bridge comprises one or more of a slide, a rail, a slot, a pivot, a hinge, a living hinge, an extension and a guide to receive the extension, a telescopic mechanism, or a rack and pinion.

Clause 6. The apparatus of any one of the preceding clauses, wherein the adjustable bridge comprises a housing and an adjustment mechanism coupled to an extension.

Clause 7. The apparatus of any one of the preceding clauses, wherein the extension is coupled to the housing and the first support.

Clause 8. The apparatus of any one of the preceding clauses, wherein the adjustment mechanism is a threaded nut.

Clause 9. The apparatus of any one of the preceding clauses, wherein the adjustment mechanism is a captured threaded nut.

Clause 10. The apparatus of any one of the preceding clauses, wherein the extension is threaded.

Clause 11. The apparatus of any one of the preceding clauses, wherein rotation of the adjustment mechanism provides nasal-temporal movement of the threaded extension.

Clause 12. The apparatus of any one of the preceding clauses, wherein the optic comprises a lens.

Clause 13. The apparatus of any one of the preceding clauses, wherein the first support comprises a left eyeglasses frame and the second support comprises a right eyeglasses frame.

Clause 14. The apparatus of any one of the preceding clauses, wherein the optic comprises one or more of a film, a layer, a beam splitter, a substrate, a wedge, or a flat layer adhered to a lens.

Clause 15. The apparatus of any one of the preceding clauses, wherein each of the plurality of projection modules further comprises: circuitry operative to activate a source of illumination in response to a control signal from a processor; and a stimulus forming element arranged to form the stimulus when illuminated by the source of illumination.

Clause 16. The apparatus of any one of the preceding clauses, wherein each of the plurality of projection modules comprises a guide element to direct light from the stimulus forming element to at least one of the plurality of optical elements.

Clause 17. The apparatus of any one of the preceding clauses, the source of illumination comprises one or more of a LED, OLED, a phosphorescent LED or a plurality of LEDs.

Clause 18. The apparatus of any one of the preceding clauses, wherein the source of illumination generates light in a specific wavelength band.

Clause 19. The apparatus of any one of the preceding clauses, wherein the source of illumination generates light in a wavelength band of no more than 25 nm.

Clause 20. The apparatus of any one of the preceding clauses, wherein the stimulus forming element comprises one or more of a mask, film, or reticle.

Clause 21. The apparatus of any one of the preceding clauses, wherein each of the plurality of projection modules comprises a light focusing element, the light focusing element comprising one or more of a lens, a spherical surface, a grin lens, a diffractive optical element, a holographic optical element, or a curved mirror.

Clause 22. The apparatus of any one of the preceding clauses, wherein the first support comprises a first plurality of projection modules that further comprise: first circuitry operative to activate a first source of illumination in response to a first control signal from a first processor; and a first stimulus forming element arranged to form the stimulus when illuminated by the first source of illumination.

Clause 23. The apparatus of any one of the preceding clauses, wherein the second support comprises a second plurality of projection modules that further comprise: second circuitry operative to activate a second source of illumination in response to a second control signal from a second processor; and a second stimulus forming element arranged to form the stimulus when illuminated by the second source of illumination.

Clause 24. The apparatus of any one of the preceding clauses, wherein the adjustable nose bridge is configured for the wearer to adjust the first support and the second support in a nasal-temporal direction.

Clause 25. The apparatus of any one of the preceding clauses, further comprising one or more adjustable pads configured for the wearer to adjust the plurality of projection optics in an inferior-superior direction with respect to one or more eyes of the wearer.

Clause 26. The apparatus of any one of the preceding clauses, wherein the one or more adjustable pads comprises a first pad to engage a first side of a nose of the wearer and a second pad to engage a second side of the nose and optionally wherein each of the pads comprises a bendable structure.

Clause 27. The apparatus of any one of the preceding clauses, further comprising one or more adjustable temple portions configured for the wearer to decrease slippage between a pair of nose pads and a nose of the wearer.

Clause 28. The apparatus of any one of the preceding clauses, further comprising one or more adjustable temple portions configured for the wearer to adjust a vertex distance between the plurality of projection modules and the eyes of the wearer.

Clause 29. The apparatus of any one of the preceding clauses, wherein one or more temple portions comprise a first temple portion to engage a first temple of the wearer and a second temple portion to engage a second temple of the wearer.

Clause 30. An apparatus to decrease a progression of a refractive error of an eye of a wearer, comprising: a first support and a second support, the first and second support each comprising: an optic, and a plurality of projection modules coupled to the optic, each of the plurality of projection modules configured to generate and direct light to form a stimulus; a bridge that couples the first support to the second support; an adjustable retainer configured to allow the wearer to move a first plurality of projection modules of the first support relative to a second plurality of projection modules of the second support to position a stimulus of the first support and a stimulus of the second support at respective locations of respective retinas of the eyes of the wearer to decrease the progression.

Clause 31. The apparatus of any one of the preceding clauses, wherein the adjustable retainer comprises the adjustable bridge that couples the first support to the second support and is configured to allow the wearer to move the plurality of projection modules.

Clause 32. The apparatus of any one of the preceding clauses, wherein the adjustable bridge is configured to independently move the first and second supports relative to a centerline of the apparatus.

Clause 33. The apparatus of any one of the preceding clauses, wherein the adjustable bridge comprises one or more of a slide, a rail, a slot, a pivot, a hinge, a living hinge, an extension and a guide to receive the extension, a telescopic mechanism, or a rack and pinion.

Clause 34. The apparatus of any one of the preceding clauses, wherein the adjustable bridge comprises a housing and an adjustment mechanism coupled to an extension.

Clause 35. The apparatus of any one of the preceding clauses, wherein the adjustable retainer is configured to move the plurality of projection modules relative to the wearable support while the optic remains substantially fixed relative to the wearable support.

Clause 36. The apparatus of any one of the preceding clauses, wherein the adjustable retainer comprises one or more of a slide, a rail, a slot, a pivot, a hinge, a living hinge, an extension and a guide to receive the extension, a telescopic mechanism, or a rack and pinion.

Clause 37. The apparatus of any one of the preceding clauses, wherein the adjustable retainer is configured to fix a position of the plurality of projection modules relative to the wearable support, and optionally wherein the retainer comprises one or more of a screw, a thumb screw, a friction pad, a brake, a hole, an extension, a guide to receive an extension, a cam, or a detent to fix the position.

Clause 38. A method of adjusting an apparatus to decrease a progression of refractive error of an eye of a wearer, the method comprising: adjusting a separation distance between a first plurality of projection optics for a first eye and a second plurality of optics for a second eye, wherein the distance is adjusted by the wearer.

Clause 39. The method of the preceding clause, wherein the first plurality of projection optics projects a first plurality of stimuli onto a first retina of the wearer and the second plurality of projection optics projects a second plurality of stimuli onto a second retina of the wearer and wherein the wearer adjusts the first plurality of projection optics and the second plurality of projection optics with an adjustable retainer while the first plurality of stimuli and the second plurality of stimuli are projected onto the first retina and the second retina, respectively.

Clause 40. An apparatus to decrease a progression of a refractive error of an eye of a wearer, comprising: an optic; a plurality of projection modules coupled to the optic, each of the plurality of projection modules operating to generate and direct light to form a stimulus; a wearable support coupled to the optic and the plurality of projection modules; and an adjustable retainer coupled to the plurality of projection modules to allow the wearer to move the plurality of projection modules relative to the wearable support to position the stimulus at a location of a retina of the eye of the wearer to decrease the progression.

Clause 41. The apparatus of any one of the preceding clauses, wherein the adjustable retainer is configured to move the plurality of projection modules relative to the wearable support while the optic remains substantially fixed relative to the wearable support.

Clause 42. The apparatus of any one of the preceding clauses, wherein the adjustable retainer is coupled to the optic and configured to move the optic and the plurality of projection modules together relative to the wearable support.

Clause 43. The apparatus of any one of the preceding clauses, wherein the adjustable retainer comprises one or more of a slide, a rail, a slot, a pivot, a hinge, a living hinge, an extension and a guide to receive the extension, a telescopic mechanism, or a rack and pinion.

Clause 44. The apparatus of any one of the preceding clauses, wherein the adjustable retainer is configured to fix a position of the plurality of projection modules relative to the wearable support, and optionally wherein the retainer comprises one or more of a screw, a thumb screw, a friction pad, a brake, a hole, an extension, a guide to receive an extension, a cam, or a detent.

Clause 45. The apparatus of any one of the preceding clauses, wherein the optic comprises a lens.

Clause 46. The apparatus of any one of the preceding clauses, wherein the lens comprises a pair of spectacles and the support comprises an eyeglass frame.

Clause 47. The apparatus of any one of the preceding clauses, wherein the adjustable retainer comprises a slide to move the pair of spectacles and the plurality of projection modules toward and away from each other in a nasal-temporal direction when worn by a wearer.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. An apparatus to decrease a progression of a refractive error of eyes of a wearer, comprising:
a first support and a second support, the first and second support each comprising:
an optic, and
a plurality of projection modules coupled to the optic, each of the plurality of projection modules operating to generate and direct light to form a stimulus; and
an adjustable bridge that couples the first support to the second support, the adjustable bridge configured to allow the wearer to move the plurality of projection modules of the first support relative to the projection modules of the second support to position the stimulus of the first support and the stimulus of the second support at respective locations of respective retinas of the eyes of the wearer to decrease the progression.

2. The apparatus of claim 1, wherein the adjustable bridge is configured to move the first support relative to a centerline of the apparatus.

3. The apparatus of claim 2, wherein the adjustable bridge is configured to move the second support relative to the centerline of the apparatus.

4. The apparatus of claim 3, wherein the adjustable bridge is configured to independently move the first and second supports relative to the centerline of the apparatus.

5. The apparatus of claim 1, wherein the adjustable bridge comprises one or more of a slide, a rail, a slot, a pivot, a hinge, a living hinge, an extension and a guide to receive the extension, a telescopic mechanism, or a rack and pinion.

6. The apparatus of claim 1, wherein the adjustable bridge comprises a housing and an adjustment mechanism coupled to an extension.

7. The apparatus of claim 6, wherein the extension is coupled to the housing and the first support.

8. The apparatus of claim 6, wherein the adjustment mechanism is a threaded nut.

9. The apparatus of claim 6, wherein the adjustment mechanism is a captured threaded nut.

10. The apparatus of claim 6, wherein the extension is threaded.

11. The apparatus of claim 10, wherein rotation of the adjustment mechanism provides nasal-temporal movement of the threaded extension.

12. The apparatus of claim 1, wherein the optic comprises a lens.

13. The apparatus of claim 12, wherein the first support comprises a left eyeglasses frame and the second support comprises a right eyeglasses frame.

14. The apparatus of claim 1, wherein the optic comprises one or more of a film, a layer, a beam splitter, a substrate, a wedge, or a flat layer adhered to a lens.

15. The apparatus of claim 1, wherein each of the plurality of projection modules further comprises:
circuitry operative to activate a source of illumination in response to a control signal from a processor; and
a stimulus forming element arranged to form the stimulus when illuminated by the source of illumination.

16. The apparatus of claim 15, wherein each of the plurality of projection modules comprises a guide element to direct light from the stimulus forming element to at least one of the plurality of optical elements.

17. The apparatus of claim 15, the source of illumination comprises one or more of a LED, OLED, a phosphorescent LED or a plurality of LEDs.

18. The apparatus of claim 15, wherein the source of illumination generates light in a specific wavelength band.

19. The apparatus of claim 15, wherein the source of illumination generates light in a wavelength band of no more than 25 nm.

20. The apparatus of claim 15, wherein the stimulus forming element comprises one or more of a mask, film, or reticle.
